(19) United States
(12) Patent Application Publication
(10) Pub. No.: US 2009/0222021 A1
(43) Pub. Date: Sep. 3, 2009

(54) Apparatus and Associated Method for Facilitating Implantation of Leads of a Cardiac Pacemaker

(76) Inventor: Raymond Jeh-Yuan Chang, San Ramon, CA (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/040,398

(22) Filed: Feb. 29, 2008

(51) Int. Cl. A61B 17/00

(52) U.S. Cl. 606/129

(57) ABSTRACT

An apparatus and method are provided for preparing a vein. A first tubular member end, defining a first lumen, is inserted into a cannulated vein, over a first guide wire. A second tubular member end, defining a second lumen, has a cutting member operably engaged therewith, and is inserted into the vein so as to intersect with and extend into the first tubular member, remotely to the first tubular member end, to establish communication between the first and second lumens, and such that a second guide wire received through the second lumen extends into the first lumen. Withdrawal of the first tubular member from the vein, over the first guide wire, causes the cutting member to longitudinally divide the first tubular member, from the intersection between the tubular members to the first tubular member end, to allow the second guide wire to pass through the division.

### BACKGROUND OF THE INVENTION

1. Field of the Invention

Embodiments of the present invention are generally directed to surgical devices and, more particularly, to an apparatus and method for facilitating implantation of leads of a cardiac pacemaker.

2. Description of Related Art

A heart is essentially a muscle that can beat over one million times during its service life (i.e., the lifetime of the entity). Normally, the heart beats in a “regular” manner, but the beats increase or decrease in response to the needs or condition of the entity's body. The heart generally beats in response to electrical signals that are provided by the entity's body and are propagated to the heart in a coordinated manner. Each electrical signal or impulse thus causes the heart (muscle) to contract in a corresponding coordinated manner to provide the pumping function for the blood in the entity's body.

As the entity ages, the complex beat regulation mechanism for the heart may become damaged or may otherwise malfunction. In such instances, the heart may fail to beat adequately or appropriately to meet the needs of the entity's body, thereby causing fainting spells or other problems, and may eventually result in sudden death. In some instances, in order for the heart to continue to beat in an appropriate manner of the body, the required electrical signals or impulses must be restored to the muscle comprising the heart. In one instance, the required electrical signals or impulses may be provided by a (cardiac) pacemaker, which generally comprises a device implanted into the body of the entity for providing the necessary electrical signal/impulse generation for the heart.

The implantation of such a pacemaker device into the body may, in some instances, be considered a “minor” surgical technique or procedure. For example, during a typical implant procedure, as shown in FIG. 1, the pacemaker device 1 is placed just under the chest tissues through a small incision. In some instances, two insulated wires or leads 2, 3 attached to the pacemaker device are used to deliver the required electrical signals/impulses, via an electrical current, to the upper right chamber (atria/atrium 4) and to the lower right chamber (ventricle 5) of the heart 6.

In order for the pacemaker wires/leads to be appropriately placed in the atria 4 and ventricle 5 of the heart 6, a vein leading to the heart 6 must first be accessed (i.e., entrance into the vein) by the surgeon. In some instances, the procedure may be facilitated by the accessed vein being relatively large. For example, the selected vein may be the subclavian vein 10, as shown in FIG. 2. To access this vein, a large hollow needle, defining a needle lumen extending therethrough, may be first inserted through the skin and into the vein (i.e., to cannulate the vein). An appropriate wire is then inserted through the needle lumen and into the vein. The needle is then withdrawn over the wire, leaving the wire in and extending along the vein. The wire thus acts as a “guide” for the insertion of other devices from outside the body into the vein. In one pacemaker insertion procedure, a relatively large cylinder or sheath is then inserted into the vein, over the wire, and the wire then removed. Upon removal of the wire, the sheath remaining in the vein provides a portal into the vein from outside the body. Any suitable, appropriately sized (i.e., small) device such as, for example, the pacemaker wire(s)/lead(s) can then be inserted into the vein from outside the body.

In some instances, each pacemaker wire/lead may be insulated with a relatively durable material. Nevertheless, due to the relative scale of the wire(s)/lead(s), the insulation may sometimes be readily damaged, for example, by being torn by a needle. The damaged insulation thus renders the wire/lead useless for the purposes of a pacemaker device. Accordingly, the relatively fragile nature of the pacemaker wire/lead may be one reason for the wire being initially used as a guide to the vein in the needle insertion procedure, instead of the pacemaker wire/lead itself.

The needle insertion step may be the most risky portion of the pacemaker implantation procedure. For example, in some instances, the needle may miss the vein entirely. In other instances, for example, the needle may be inadvertently inserted too far, piercing completely through the vein, extending to and puncture the lung, or otherwise causing an emergency situation. Since two pacemaker wires or leads are typically utilized by a pacemaker device (one wire/lead extending to the upper chamber or atrium 4, and one wire/lead extending to the lower chamber or ventricle 5), two separate hollow needle insertions are generally used during the pacemaker implantation procedure. However, doubling the number of needle insertions may also correspondingly double the risk of complications.

Thus, there exists a need for an apparatus and method for facilitating implantation of leads of a cardiac pacemaker that eliminates or minimizes the risk of complications for the entity/patient during a pacemaker implantation procedure and/or the risk of damage to the leads of the pacemaker device during the implantation process.

### BRIEF SUMMARY OF THE INVENTION

The above and other needs are met by aspects of the present invention which, in one embodiment, provides an apparatus for preparing a vein leading to a heart for insertion of leads of a cardiac pacemaker therein. Such an apparatus comprises a first tubular member defining a first lumen and having an end. The end of the first tubular member is adapted to be inserted into a cannulated vein, over a first guide wire inserted into the vein, so as to extend along the vein over the first guide wire. A second tubular member defines a second lumen and has an end with a cutting member operably engaged therewith. The end of the second tubular member is configured to be inserted into the vein proximate to the first tubular member. The second tubular member is configured to cooperate with the first tubular member such that the end of the second tubular member intersects with and extends into the first tubular member, remotely with respect to the end of the first tubular member, to establish communication between the first and second lumens such that a second guide wire received through the second lumen extends from the second lumen into the first lumen along the first guide wire. The second tubular member is further configured such that withdrawal of the first tubular member from the vein, over the first guide wire, causes the first tubular member to be longitudinally divided by the cutting member, from the intersection between the first and second tubular members to the end of the first tubular member, so as to allow the second guide wire to pass through the longitudinal division.

Another aspect of the present invention provides a method of preparing a vein leading to a heart for insertion of leads of a cardiac pacemaker therein. Such a method comprises cannulating the vein and inserting a first guide wire therein such that the first guide wire extends along the vein. An end of a first tubular member, defining a first lumen, is then inserted into the cannulated vein over the first guide wire, wherein the first lumen is configured to receive and extend along the first guide wire. An end of a second tubular member, defining a second lumen and including a cutting member operably engaged with the end thereof, is then inserted into the vein proximate to the first tubular member, such that the end of the second tubular member intersects with and extends into the first tubular member, remotely with respect to the end of the first tubular member, to establish communication between the first and second lumens. A second guide wire is inserted through the second lumen such that the second guide wire extends from the second lumen into the first lumen along the first guide wire. The first tubular member is then withdrawn from the vein, over the first guide wire, such that the first tubular member is longitudinally divided by the cutting member, from the intersection between the first and second tubular members to the end of the first tubular member, so as to allow the second guide wire to pass through the longitudinal division. The second tubular member is withdrawn from the vein, over the second guide wire, such that the first and second guide wires remain within and extend along the vein. First and second sheaths, defining respective first and second sheath lumens, are then inserted into the vein over the respective first and second guide wires, wherein the first and second sheath lumens are configured to respectively receive and extend along the first and second guide wires disposed within and extending along the vein. The first and second guide wires are then withdrawn from the vein through the respective first and second sheath lumens, and first and second leads of the cardiac pacemaker are then inserted through the respective first and second sheath lumens, such that the first and second leads extend to the heart

Embodiments of the present invention thus provide an apparatus and method that can be used after the first needle insertion in the vein leading to the heart during a cardiac pacemaker implantation procedure using, for example, the Seldinger technique, wherein such an apparatus and method facilitates the procedurally-required second needle insertion into the vein, while reducing or minimizing the risk of, for instance, the second needle insertion missing the vein or undesirably extending completely through the vein, and thereby avoiding or minimizing the risk of inadvertent trauma to surrounding tissues or the lung of the patient during the cardiac pacemaker implantation procedure. As such, aspects of the present invention provide distinct advantages as otherwise disclosed herein.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 schematically illustrates a cardiac pacemaker implanted into a patient;

FIG. 2 schematically illustrates a vein leading to the heart, cannulated with a first guide wire using, for example, the Seldinger technique;

FIG. 3A schematically illustrates a first tubular member of a vein preparation apparatus for insertion of pacemaker leads, according to one aspect of the present invention, extending over the first guide wire for insertion into the vein;

FIG. 3B schematically illustrates an engagement mechanism operably engaged between the first and second tubular members of a vein preparation apparatus for insertion of pacemaker leads, according to one aspect of the present invention;

FIG. 4 schematically illustrates a first tubular member of a vein preparation apparatus for insertion of pacemaker leads, according to one aspect of the present invention, extending over the first guide wire and inserted into the vein, with a second tubular member, according to one aspect of the present invention, also inserted into the vein in cooperation with the first tubular member via the engagement mechanism operably engaged therebetween;

FIG. 5 schematically illustrates a vein preparation apparatus for insertion of pacemaker leads, according to one aspect of the present invention, with the second tubular member inserted into the vein and extending into the first tubular member so as to establish communication between first and second lumens defined by the respective first and second tubular members;

FIGS. 6A-6C schematically illustrate an insertion sequence of the second tubular member of a vein preparation apparatus for insertion of pacemaker leads, according to one aspect of the present invention, being inserted into the vein and extending into the first tubular member so as to establish communication between first and second lumens defined by the respective first and second tubular members;

FIG. 6D schematically illustrates a second guide wire inserted into the vein through the established communication between the first and second tubular members of a vein preparation apparatus for insertion of pacemaker leads, according to one aspect of the present invention;

FIG. 7 schematically illustrates a releasable locking mechanism associated with the engagement mechanism operably engaged between the first and second tubular members, according to one embodiment of the present invention, for determining relative movement between the first and second tubular members;

FIG. 8 schematically illustrates the insertion of a second guide wire through the second tubular member and into the first tubular member, so as to extend along the vein with the first guide wire, due to the communication between the first and second lumens upon the second tubular member being inserted into the first tubular member, according to one embodiment of the present invention;

FIG. 9 schematically illustrates one embodiment of a first tubular member of a vein preparation apparatus for insertion of pacemaker leads, according to one aspect of the present invention, wherein the first tubular member includes a receptor site and a withdrawal path;

FIG. 10 schematically illustrate a cutting member associated with the end of the second tubular member, and configured to longitudinally divide the first tubular member from the intersection between the first and second tubular members to the end of the first tubular member, upon withdrawal of the first tubular member from the vein, according to one embodiment of the present invention;

FIG. 11 schematically illustrate the withdrawal of the first tubular member from the vein, separately from the second tubular member, upon the release of the locking mechanism, so as to allow relative movement between the first and second tubular members, according to one embodiment of the present invention;

FIG. 12 schematically illustrates the longitudinal division of the first tubular member by the cutting mechanism associated with the second tubular member upon withdrawal of the first tubular member from the vein, according to one embodiment of the present invention;

FIG. 13 schematically illustrates the first and second guide wires remaining within the vein, the second guide wire being disposed within the second tubular member, after withdrawal of the first tubular member from the vein, according to one embodiment of the present invention;

FIG. 14 schematically illustrates the first and second guide wires remaining within the vein, after withdrawal of the first and second tubular members, according to one embodiment of the present invention, wherein the first and second guide wires disposed within the vein being configured and prepared to receive appropriate and respective first and second sheaths thereover for facilitating insertion of the pacemaker leads therethrough into the heart; and

FIGS. 15-18 schematically illustrate the pacemaker implantation process, whereby respective sheaths are inserted into the vein over the first and second guide wires, the first and second guide wires then withdrawn, the leads of the pacemaker device then inserted through the sheaths into the vein, and the sheaths then withdrawn and the pacemaker device attached to the leads extending into the vein and to the heart.

### DETAILED DESCRIPTION OF THE INVENTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

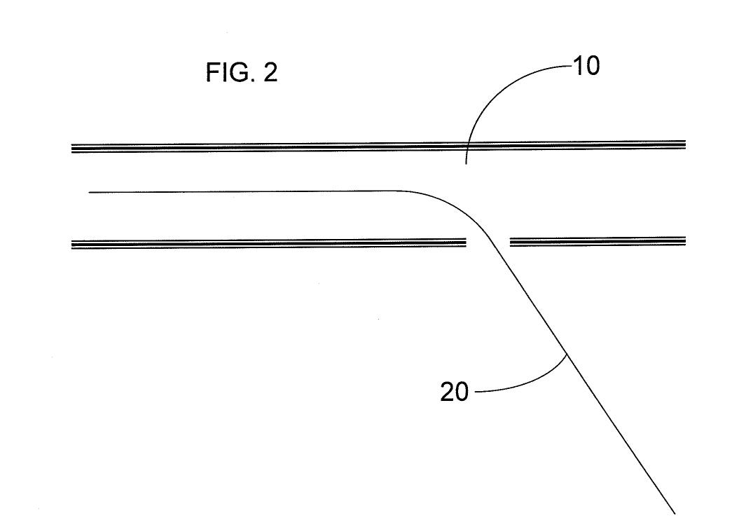

FIG. 2 schematically illustrates a vein leading to the heart 6, such as the subclavian vein 10. Using, for example, the Seldinger technique, as will be appreciated by one skilled in the art, the subclavian vein 10 may be cannulated with a hollow needle so as to insert a first guide wire 20 therein. Upon removal or withdrawal of the hollow needle, the first guide wire 10 remains within the subclavian vein 10.

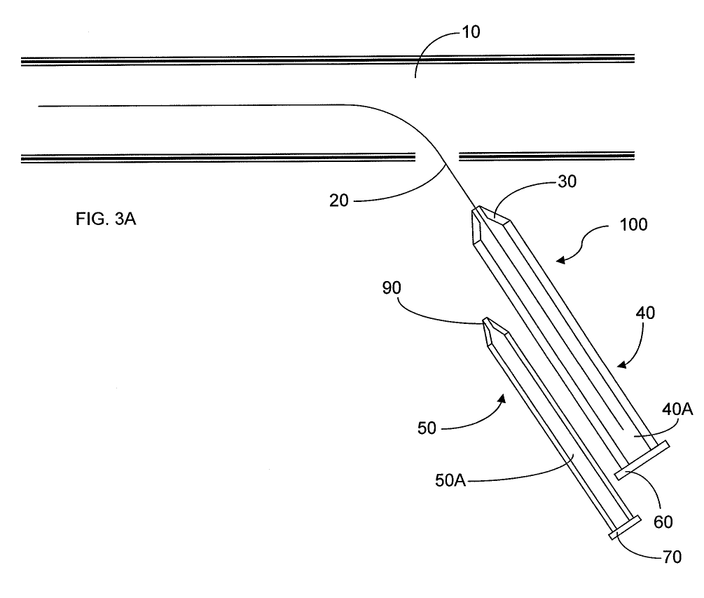

FIG. 3A schematically illustrates a vein preparation apparatus 100 according to one embodiment of the present invention. Such a vein preparation apparatus 100 may be particularly configured, for example, for facilitating insertion of operative wires or leads 2, 3 of a pacemaker device 1, according to one aspect of the present invention. The vein preparation apparatus 100 comprises, for instance, first and second tubular members 40, 50. One skilled in the art will appreciate that each of the first and second tubular members 40, 50 defines a respective lumen 40A, 50A extending along the length thereof. Each of the first and second tubular members 40, 50 includes respective first and second insertion ends 30, 90 for insertion into the vein 10, as further discussed herein. Opposite the first and second insertion ends 30, 90, the respective first and second tubular members 40, 50 each include a valve mechanism 60, 70. Each valve mechanism 60, 70 is configured to allow a guide wire, such as the first guide wire 20, to pass therethrough, but preferably and substantially prevents the flow of blood or other fluids therethrough. For example, each valve mechanism 60, 70 may comprise a self-sealing diaphragm or any other suitable “one-way” valve. As shown, the first tubular member 40 of the vein preparation apparatus 100 is first inserted over the first guide wire 20 through the insertion end 30 thereof, such that the first guide wire 20 extends from the vein 10 through the first lumen 40A defined by the first tubular member 40.

FIG. 3B schematically illustrates that each of the first and second tubular members 40, 50 may include complementary and interacting first and second portions 110, 120 of an engagement mechanism 130 operably engaged between the first and second tubular members 40, 50 of the vein preparation apparatus 100. In one aspect, the first and second portions 110, 120 of the engagement mechanism 130 are particularly configured to effect a sliding relation between the first and second tubular members 40, 50. That is, the engagement between the first and second portions 110, 120 may allow only relative longitudinal movement and/or substantially parallel movement between the first and second tubular members 40, 50. For example, the first portion 110 engaged with the first tubular member 40 may be configured as a “T” shaped member, while the second portion 120 engaged with the second tubular member 50 may be configured as a “C” shaped channel appropriate for receiving the “T” shaped member therein. The engagement mechanism 130, thus configured, allows the first and second tubular members 40, 50 to “slide” relative to each other, which may be useful, for example, when one tubular member is inserted into or withdrawn from the vein 10 separately from the other tubular member, as discussed further herein. In some instances, a friction-reducing substance (i.e., Teflon™, polytetrafluouroethylene, or any other suitable friction-reducing substance) may be applied to either or both of the first and second portions 110, 120, so as to facilitate the sliding relation therebetween.

According to one aspect of the present invention, the first tubular member 40 may be configured to be flexible and formed of a suitable and appropriate material for insertion into a patient's body, as will be appreciated by one skilled in the art. For example, the first tubular member 40 may be comprised of a metallic material, a polymeric material, a composite material, or any other suitable and appropriate material or combinations thereof. Further, the end 30 of the first tubular member 40 is particularly configured to be capable of insertion into the vein 10 through the opening therein formed by the hollow needle originally used to cannulate the vein 10. That is, one skilled in the art will appreciate that the end 30 may not necessarily be “sharp” since the first tubular member 40 is being inserted into the vein 10 through an existing hole. However, the end 30 may also be “sharp” (i.e., capable of independently piercing the vein 10) where necessary of desirable.

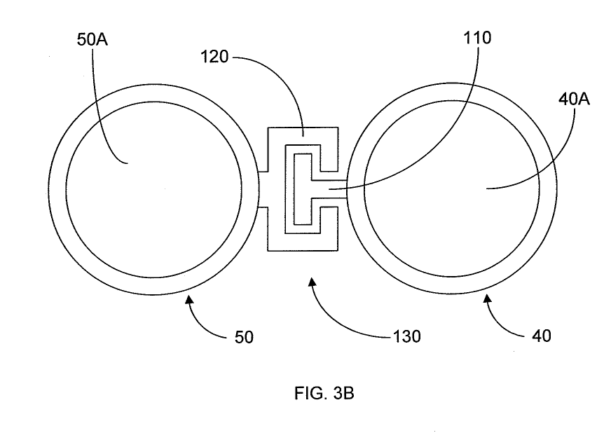

Once the first tubular member 40 is installed over the first guide wire 20 and the end 30 thereof inserted into the vein 10, as shown in FIG. 4, the first tubular member 40 may be at least partially advanced over the first guide wire 20 and along the vein 10 toward the heart 6. As a result, the first guide wire 20 may extend through the first lumen 40A to pierce or otherwise interact with the valve mechanism 60 to extend therethrough, outwardly of the first tubular member 40. In such instances, the first tubular member 40 may be inserted into the vein 10 at an acute angle with respect thereto. As such, the first tubular member 40 must bend (i.e., must be sufficiently flexible to bend) to follow the first guide wire 20 along the vein 10. Accordingly, in some aspects, the first tubular member 40 may be configured to be selectively flexible or to otherwise be flexible only in a particular direction. For example, the first tubular member 40 may be configured to be flexible in a direction that intersects the projected travel path of the second tubular member 50, as determined by the interaction between the first and second portions 110, 120 of the engagement mechanism 130 (i.e., substantially parallel to the first tubular member 40). As such, the first tubular member would have to be rotated about the longitudinal axis thereof in order for the first tubular member 40 to follow the first guide wire 20 along the vein 10 upon insertion of the first tubular member 40 into the vein 10. In doing so, the appropriate positioning of the selective flexible first tubular member 40 may thus align the second tubular member 50 on an intersecting path with the vein 10. Thus, one such benefit of the vein preparation apparatus 100 may be the alignment of the second tubular member 50 with the vein so as to reduce, minimize, or eliminate the risk of missing the vein 10 upon insertion of the second tubular member 50.

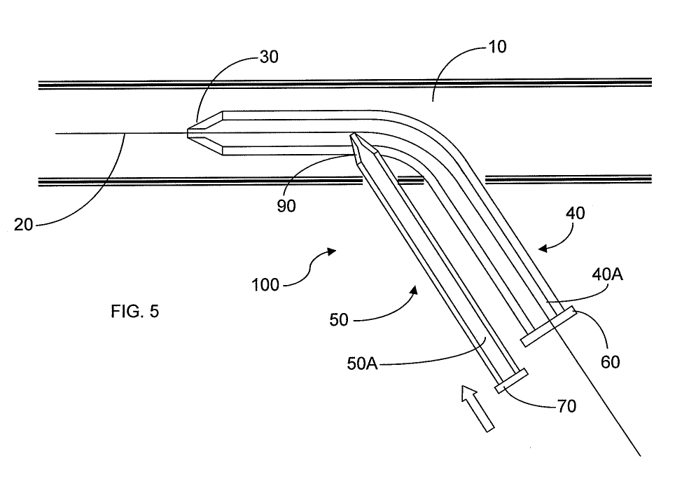

FIG. 4 further schematically illustrates the second tubular member 50, thus aligned with the vein 10 through the insertion of the first tubular member 40 and the configuration of the engagement mechanism 130, being advanced toward and inserted into the vein 10 in proximity to the first tubular member 40 (as also shown in FIG. 6A). That is, in some instances, the second tubular member 50 merely needs to be advanced toward the vein 10 for insertion therein, since the second tubular member 50 is already aligned with the vein 10 via the engagement mechanism 130 and possibly the configuration of the first tubular member 40 when inserted into the vein 10, as previously discussed. In one aspect, the second tubular member 50 may be configured to be substantially rigid and formed of a suitable and appropriate material for insertion into a patient's body, as will be appreciated by one skilled in the art. For example, the second tubular member 50 may be comprised of a metallic material, a polymeric material, a composite material, or any other suitable and appropriate material or combinations thereof. Further, the end 90 of the second tubular member 50 is particularly configured to be capable of piercing into the vein 10, and thus may necessarily be “sharp” since there is no pre-existing hole in the vein 10 through which the second tubular member 50 can be inserted. In one particular instance, the second tubular member 50 may essentially be configured as a hollow needle.

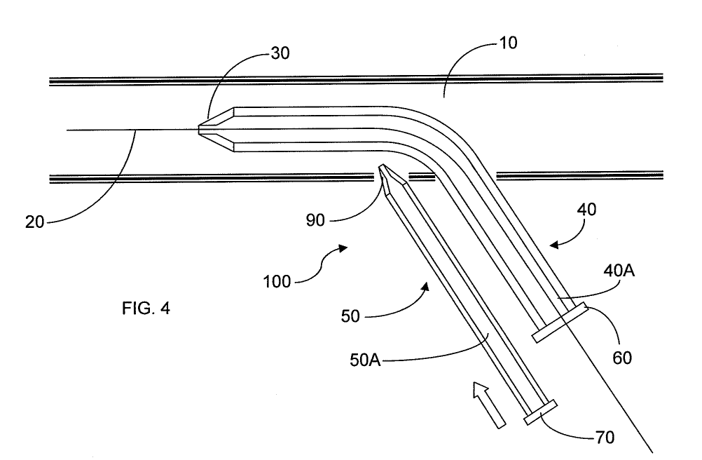

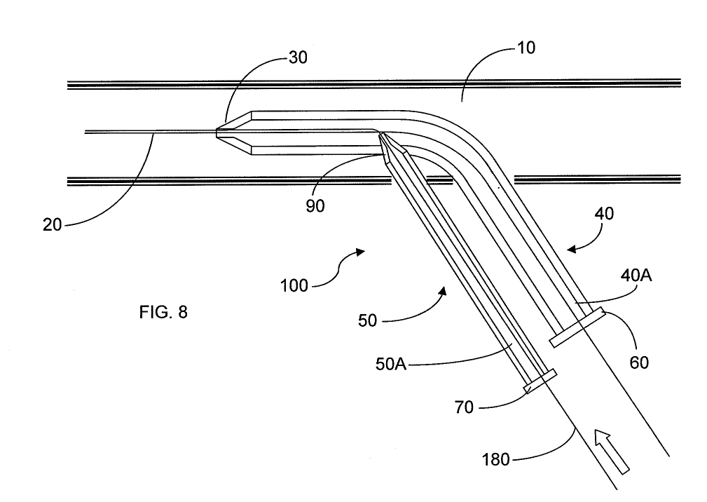

As shown in FIGS. 5, 6B, and 6C, once the second tubular member 50 pierces and is inserted into the vein 10, the second tubular member 50 is advanced into intersection with the first tubular member 40 extending along the vein 10 over the first guide wire 20. In one instance, the end 90 of the second tubular member 50 is advanced so as to pierce and enter the first tubular member 40 so as to establish communication between the respective first and second lumens 40A, 50A defined by the respective first and second tubular members 40, 50. In another instance, the first tubular member 40 may be configured to include or otherwise define a receptor site 95 (see, e.g., FIGS. 6A and 9) at or about a predetermined intersection with the end 90 of the second tubular member 50. For example, the receptor site 95 may comprise a relatively thinner section of the wall of the first tubular member 40, or other factor for promoting or facilitating the insertion of the end 90 of the second tubular member 50 into the first tubular member 40.

In some aspects, the travel of the second tubular member 50 may be limited, for example, by the engagement mechanism 130 and/or a locking mechanism 140 associated with the engagement mechanism 130 and/or the first and second tubular member 40, 50. For instance, the first and/or second portion 110, 120 of the engagement mechanism 130 may be configured to include a stop member (not shown) that limits the longitudinal travel of the second tubular member 50 with respect to the first tubular member 40. In such a manner, the second tubular member 50 may be advanced into the vein 10 and into the first tubular member 40, but may not be permitted to extend through the first tubular member 40 across the first lumen 40A (i.e., the travel of the second tubular member 50 may be limited such that the end 90 does not pierce through the opposite side of the vein 10).

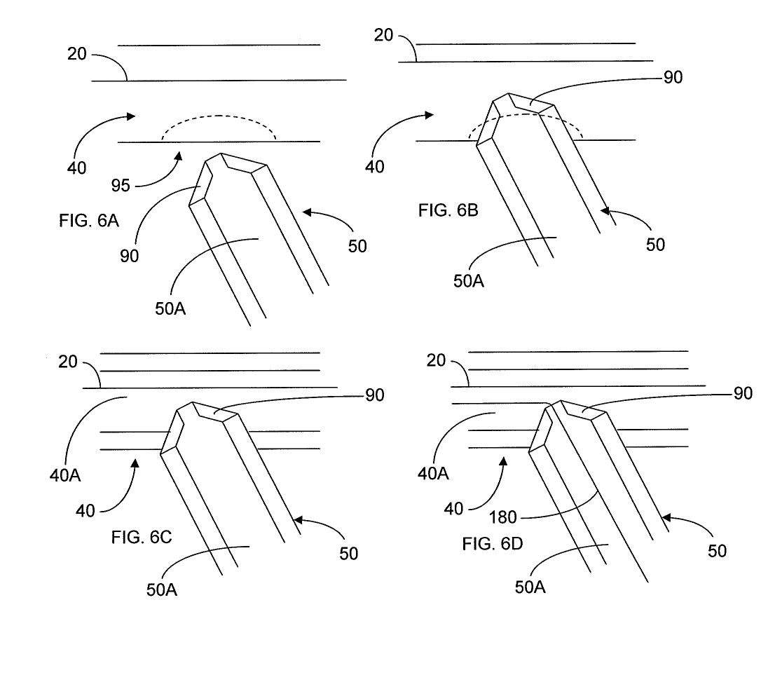

FIG. 7 further schematically illustrates that the travel limitation and/or prevention of relative movement between the first and second tubular members 40, 50 may also be accomplished with the releasable locking mechanism 140 associated with the engagement mechanism 130 and/or the first and second tubular members 40, 50. In one aspect, one of the first and second tubular members 40, 50 may include a protrusion 150 extending therefrom toward the other of the first and second tubular members 40, 50, with the first and second tubular members 40, 50 being adjacently disposed through the engagement mechanism 130. In such an aspect, the other of the first and second tubular members 40, 50 may include a releasable lock receptacle having a stop member 160 and a release member 170. Accordingly, as the second tubular member 50 is advanced into the vein 10 and then into the first tubular member 40 therein, the protrusion 150 longitudinally advances to engage the release member 170 which, in some instances, may be configured to be resilient (i.e., a “spring tab”). Further advancement of the second tubular member 50 results in the depression of the release member 170, as the protrusion 150 passes thereby toward the stop member 160. The travel of the second tubular member 50 may then be limited or stopped when the protrusion 150 encounters the stop member 160, at which point, the release member 170 resiliently rebounds, thereby “locking” the protrusion between the stop member 160 and the release member 170. At this point, according to one aspect, the end 90 of the second tubular member 50 may have pierced the first tubular member 40 to establish communication between the first and second lumens 40A, 50A.

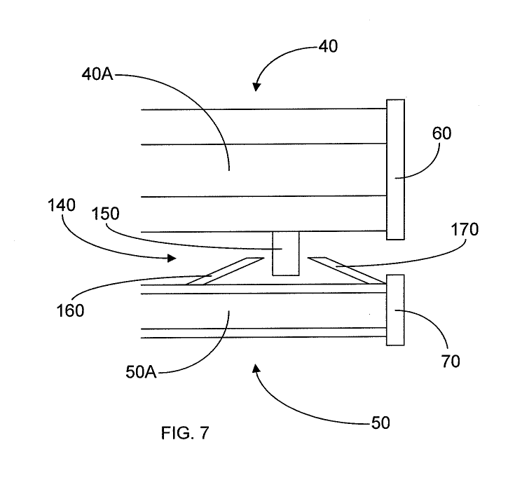

FIGS. 6D and 8 schematically illustrates the insertion of a second guide wire 180 through the valve mechanism 70, and then through the second lumen 50A defined by the second tubular member 50. Upon further advancement of the second guide wire 180, the second guide wire 180 extends through the intersection between the first and second tubular members 40, 50 (i.e., via the communication between the first and second lumens 40A, 50A) and into the lumen 40A defined by the first tubular member 40. In this manner, the second guide wire 180 may be advanced to extend along the vein 10 along with the first guide wire 20. At this point, both the first and second guide wires 20, 180 may extend through the end 30 of the first tubular member 40 toward the heart 6. In this manner, the “second cannulation” of the vein 10 is essentially complete, whereby the risk of missing the vein 10 upon insertion of the second tubular member 50 is thus further reduced, minimized, or eliminated. However, the first and second tubular members 40, 50 may not necessarily be configured to facilitate insertion of the operative wires or leads 2, 3 of the pacemaker device 1. Accordingly, aspects of the present invention further involve removing the first and second tubular members 40, 50 from the vein, while leaving the first and second guide wires 20, 180 intact and disposed with the vein 10.

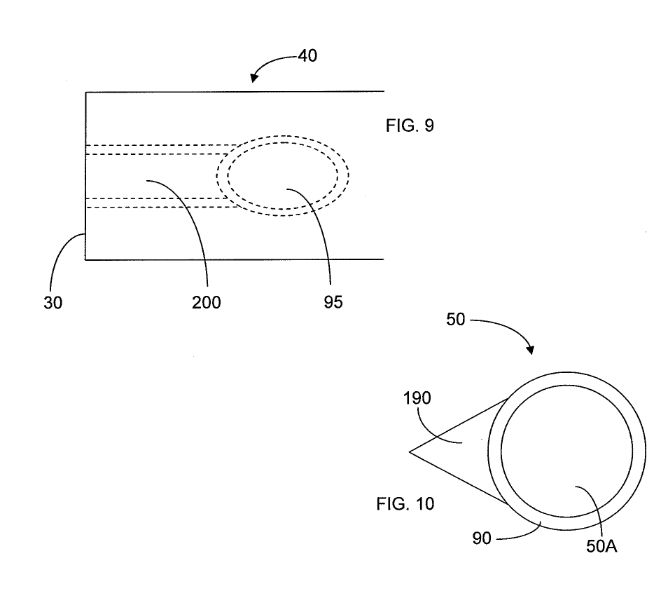

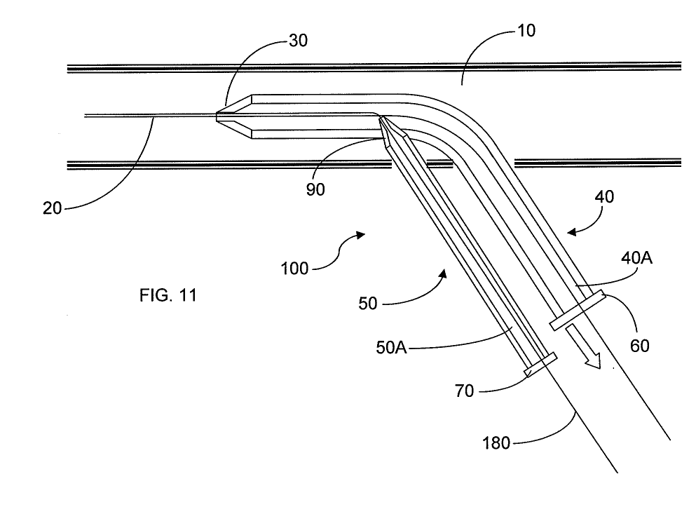

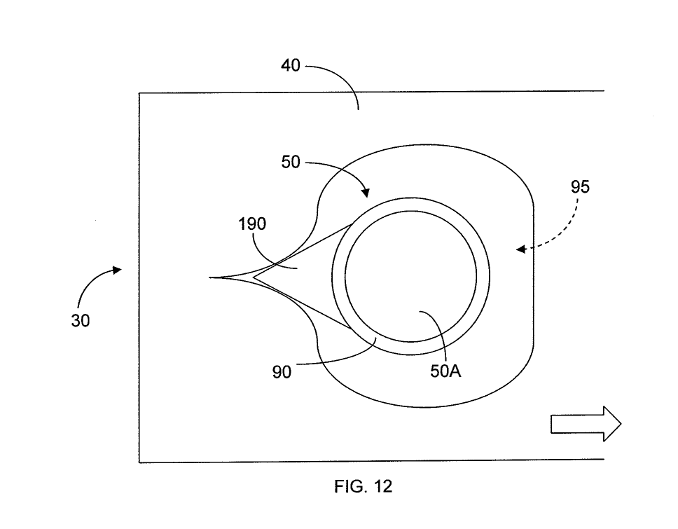

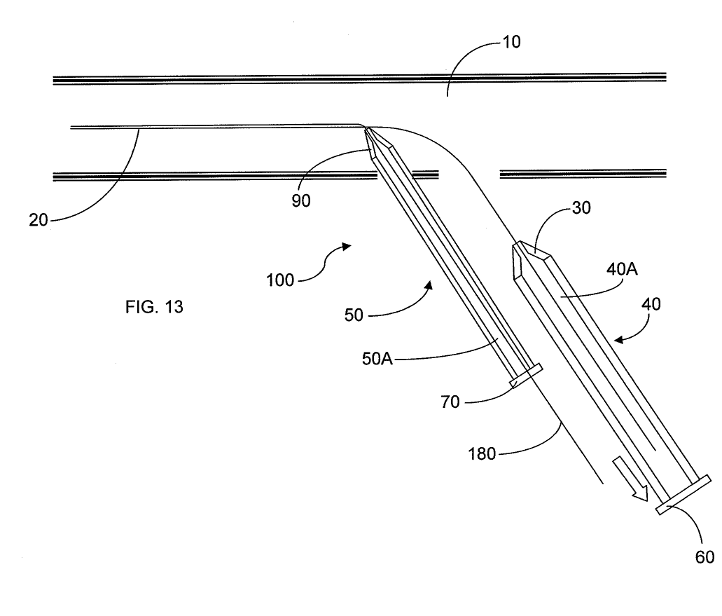

FIGS. 10-14 thus schematically illustrate a configuration for allowing the withdrawal of the first and second tubular members 40, 50 from the vein 10, while the first and second guide wires 20, 180 remain within the vein 10. In such a configuration, the second tubular member 50 may further include a cutting member 190 associated with the end 90 of the second tubular member 50, as shown in FIG. 10, wherein such a cutting member 190 may be configured to extend into the first tubular member 40 upon appropriate insertion of the end 90 of the second tubular member 60 therein. The cutting member 190 may be sufficiently “sharp” such that, upon withdrawal of the first tubular member 40 from the vein 10, as shown in FIG. 11 (while leaving the first guide wire 20 disposed within the vein 10), the cutting member 190 longitudinally divides (i.e., cuts) the first tubular member 40 from the intersection between the first and second tubular members 40, 50 (i.e., at the receptor site 95) to the end 30 of the first tubular member 40, as shown in FIG. 12. That is, upon withdrawal of the first tubular member 40 from the vein 10, the cutting member 190 associated with the end 90 of the second tubular member 50 cuts or slices the first tubular member 40 up to and through the end 30 thereof such that the first tubular member 40 is withdrawn around the second tubular member 50 and the second guide wire 180 extending therethrough. In this manner, the second tubular member 50 and the second guide wire 180 extending therethrough remain in the vein 10 alongside the first guide wire 20, when the first tubular member 40 is withdrawn from the vein, without bending or damaging the second guide wire 180 or causing trauma to the vein 10, as shown in FIG. 13.

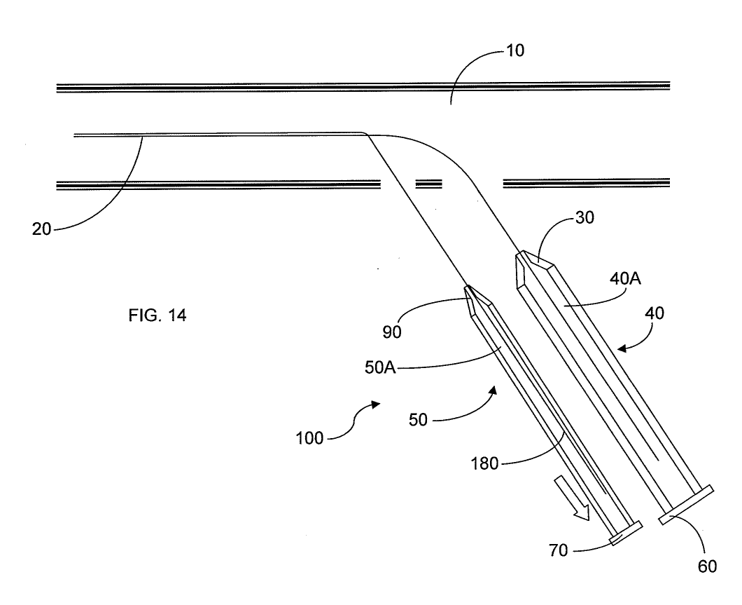

In some instances, in order to further facilitate the first tubular member 40 withdrawal process, the first tubular member 40, from the intersection between the first and second tubular members 40, 50 (i.e., at the receptor site 95) to the end 30 of the first tubular member 40, may be configured to define a withdrawal path 200, as shown in FIG. 9. The withdrawal path 200 may comprise, for example, a relatively thinner section of the wall of the first tubular member 40, or other factor for promoting or facilitating the longitudinal division of the first tubular member 40 by the cutting member 190 associated with the second tubular member 50. Further, one skilled in the art will appreciate from the disclosure herein that, in order to withdraw the first tubular member 40 from the vein 10, the locking mechanism 140 shown in FIG. 7 must first be released, for example, by depressing the release member 170 so as to allow the protrusion 150 to be removed from between the stop member 160 and the release member 170. Release of the locking mechanism 140 thus allows relative movement between the first and second tubular members 40, 50 such that the first tubular member 40 can be withdrawn from the vein 10 separately from the second tubular member 50, as shown in FIG. 13. Removal or withdrawal of the first tubular member 40 from the vein 10 leaves the first and second guide wires 20, 180 remaining within the vein 10, with the second guide wire 180 being disposed within and extending through the second tubular member 50, as shown in FIG. 13. As such, withdrawal of the second tubular member 50 leaves both the first and second guide wires 20, 180 extending into and along the vein 10, as shown in FIG. 14.

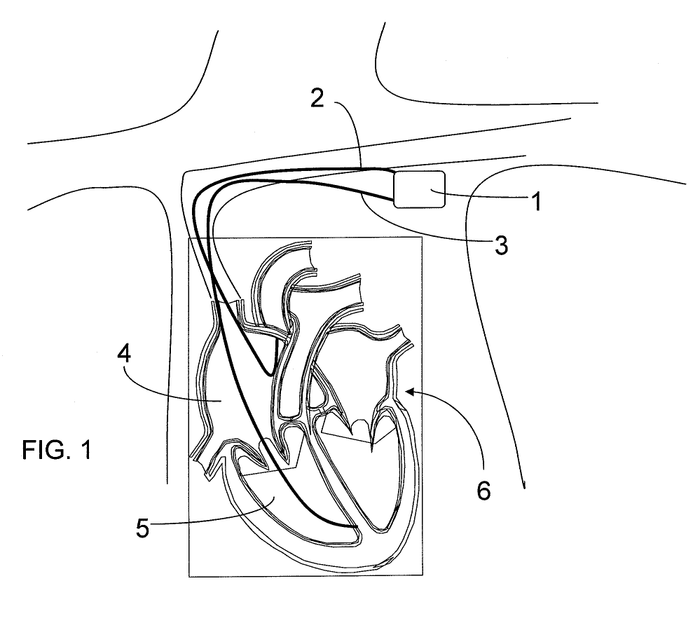

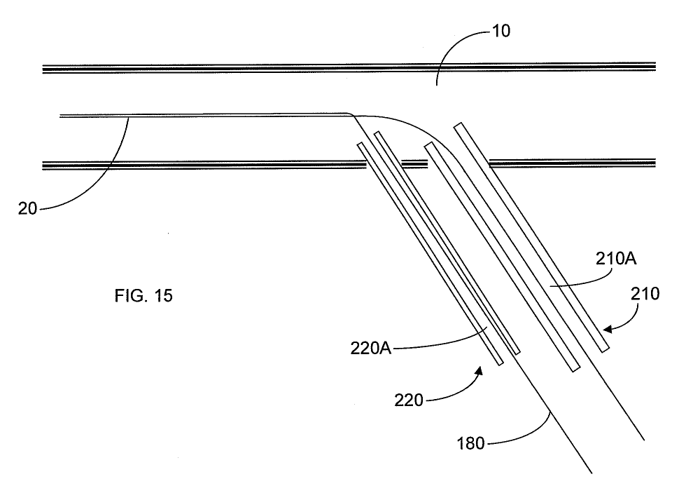

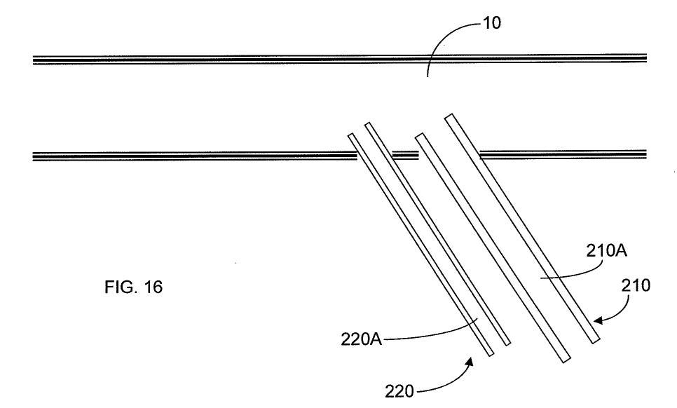

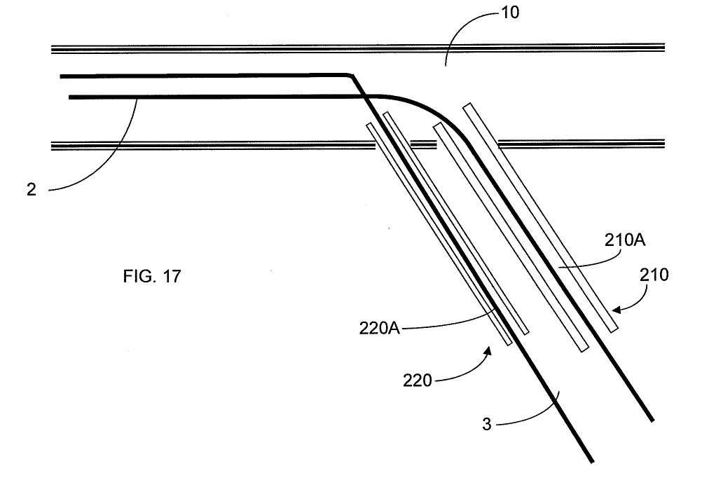

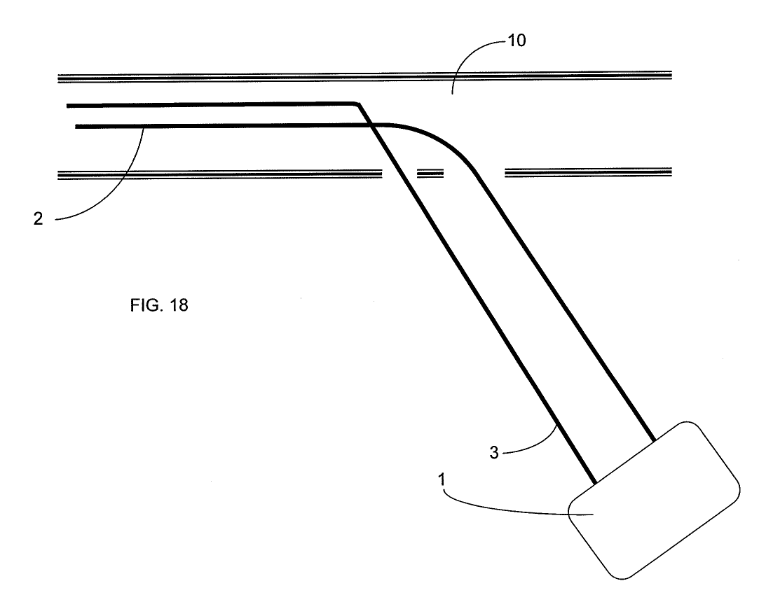

The first and second guide wires 20, 180 remaining within the vein 10, after withdrawal of the first and second tubular members 40, 50, are thus configured to further facilitate the completion of the cardiac pacemaker implantation procedure, as will be appreciated by one skilled in the art. More particularly, each of the first and second guide wires 20, 180 may receive appropriate and respective first and second sheaths 210, 220 thereover such that the first and second sheaths 210, 220 extend into the vein 10 and toward the heart 6, as shown in FIG. 15. The first and second guide wires 20, 180 can then be withdrawn from the vein 10, leaving the first and second sheaths 210, 220 intact and disposed within the vein 10, as shown in FIG. 16. The operative wires 2, 3, of the pacemaker 1 can then be inserted into and through respective sheath lumens 210A, 220A defined by the first and second sheaths 210, 220 and into the vein 10, as shown in FIG. 17, at which point, the leads 2, 3, may be directed by the surgeon to the appropriate locations within the heart 6. Once the leads 2, 3 are appropriately placed, the first and second sheaths 210, 220 are then withdrawn from the vein 10 and the ends of the leads 2, 3 external to the vein attached to the pacemaker device 1 configured to generate the electrical signals/impulses that are transmitted by the leads 2, 3 to the heart 6, as shown in FIGS. 1 and 18.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. An apparatus for preparing a vein leading to a heart for insertion of leads of a cardiac pacemaker therein, comprising:
a first tubular member defining a first lumen and having an end, the end of the first tubular member being adapted to be inserted into a cannulated vein, over a first guide wire inserted into the vein, so as to extend along the vein over the first guide wire; and
a second tubular member defining a second lumen and having an end with a cutting member operably engaged therewith, the end of the second tubular member being configured to be inserted into the vein proximate to the first tubular member, the second tubular member being configured to cooperate with the first tubular member such that the end of the second tubular member intersects with and extends into the first tubular member, remotely with respect to the end of the first tubular member, to establish communication between the first and second lumens such that a second guide wire received through the second lumen extends from the second lumen into the first lumen along the first guide wire, the second tubular member being further configured such that withdrawal of the first tubular member from the vein, over the first guide wire, causes the first tubular member to be longitudinally divided by the cutting member, from the intersection between the first and second tubular members to the end of the first tubular member, so as to allow the second guide wire to pass through the longitudinal division.

2. An apparatus according to claim 1 wherein the second tubular member is configured to be inserted into the vein separately with respect to the first tubular member.

3. An apparatus according to claim 1 wherein each of the first and second tubular members further comprises respective first and second complementary portions of an engagement mechanism, the engagement mechanism being configured to guide the second tubular member with respect to the first tubular member as the second tubular member is inserted into the vein, and to secure the second tubular member with respect to the first tubular member upon the end of the second tubular member intersecting with and extending into the first tubular member, remotely with respect to the end of the first tubular member, to establish communication between the first and second lumens.

4. An apparatus according to claim 3 wherein the engagement mechanism further comprises a locking mechanism configured to at least one of substantially prevent relative movement between the first and second tubular members, and limit extension of the second tubular member into the first tubular member so as to prevent the second tubular member from extending through the first tubular member across the first lumen.

5. An apparatus according to claim 4 wherein the locking mechanism is configured to be releasable so as to at least one of allow relative movement between the first and second tubular members, and allow the first and second tubular members to be withdrawn from the vein discretely of each other.

6. An apparatus according to claim 3 further comprising a friction-reducing substance operably engaged with at least one of the first and second portions of the engagement mechanism for facilitating a sliding relation therebetween.

7. An apparatus according to claim 1 wherein the first tubular member is flexible and the second tubular member is rigid.

8. An apparatus according to claim 7 wherein the first and second tubular members are further configured to be inserted into the vein in parallel relation to each other, and wherein the first tubular member is further configured to flex in a direction intersecting the second tubular member upon insertion into the vein.

9. An apparatus according to claim 1 wherein the first tubular member defines a receptor site, remotely with respect to the end of the first tubular member, configured to receive the end of the second tubular member therethrough so as to define the intersection between the first and second tubular members and to establish communication between the first and second lumens.

10. An apparatus according to claim 9 wherein the first tubular member defines a withdrawal path extending from the receptor site to the end of the first tubular member, the withdrawal path being configured to be longitudinally divided by the cutting member, upon withdrawal of the first tubular member from the vein, so as to allow the second guide wire extending through the second tubular member to pass through the longitudinal division and remain in the vein.

11. An apparatus according to claim 1 wherein the end of the second tubular member is configured to be capable of piercing the first tubular member to establish the communication between the first and second lumens.

12. An apparatus according to claim 1 wherein the second tubular member is configured as a hollow needle.

13. A method of preparing a vein leading to a heart for insertion of leads of a cardiac pacemaker therein, comprising:
cannulating the vein and inserting a first guide wire therein such that the first guide wire extends along the vein;
inserting an end of a first tubular member, defining a first lumen, into the cannulated vein over the first guide wire, the first lumen being configured to received and extend along the first guide wire;
inserting an end of a second tubular member, defining a second lumen and including a cutting member operably engaged with the end thereof, into the vein proximate to the first tubular member such that the end of the second tubular member intersects with and extends into the first tubular member, remotely with respect to the end of the first tubular member, to establish communication between the first and second lumens;
inserting a second guide wire through the second lumen such that the second guide wire extends from the second lumen into the first lumen along the first guide wire;
withdrawing the first tubular member from the vein, over the first guide wire, such that the first tubular member is longitudinally divided by the cutting member, from the intersection between the first and second tubular members to the end of the first tubular member, so as to allow the second guide wire to pass through the longitudinal division; and
withdrawing the second tubular member from the vein, over the second guide wire, such that the first and second guide wires remain within and extend along the vein.

14. A method according to claim 13 further comprising:
inserting first and second sheaths, defining respective first and second sheath lumens, into the vein over the respective first and second guide wires, the first and second sheath lumens being configured to respectively receive and extend along the first and second guide wires disposed within and extending along the vein; and
withdrawing the first and second guide wires from the vein through the respective first and second sheath lumens.

15. A method according to claim 14 further comprising inserting first and second leads of the cardiac pacemaker through the respective first and second sheath lumens such that the first and second leads extend to the heart.

16. A method according to claim 13 wherein inserting the end of the second tubular member further comprises inserting the end of the second tubular member into the vein separately with respect to inserting the end of the first tubular member.

17. A method according to claim 13 wherein each of the first and second tubular members further comprises respective first and second complementary portions of an engagement mechanism, and inserting the end of the second tubular member further comprises inserting the end of the second tubular member such that the first and second portions of the engagement mechanism cooperate to guide the second tubular member with respect to the first tubular member as the second tubular member is inserted into the vein.

18. A method according to claim 17 further comprising at least one of limiting the insertion of the end of the second tubular member into the first tubular member so as to prevent the second tubular member from extending through the first tubular member across the first lumen, and substantially preventing relative movement between the first and second tubular members to secure the second tubular member with respect to the first tubular member upon the end of the second tubular member intersecting with and extending into the first tubular member, with a locking mechanism operably engaged with the engagement mechanism.

19. A method according to claim 18 further comprising releasing the locking mechanism so as to allow relative movement between the first and second tubular members, wherein withdrawing the first tubular member from the vein further comprises withdrawing the first tubular member from the vein discretely of the second tubular member.
